(19) United States
(12) Patent Application Publication
(10) Pub. No.: US 2026/0345854 A1
(43) Pub. Date: Nov. 5, 2026

(54) DEVICE FOR IONIZING AIR

(71) Applicant: HERRMANN AG, Poesing (DE)

(72) Inventor: Johannes HERRMANN, Cham (DE)

(21) Appl. No.: 19/668,568

(22) Filed: May 5, 2026

(30) Foreign Application Priority Data
May 5, 2025 (DE) 20 2025 102 452.7

(51) Int. Cl. A61L 9/22; A61L 2/02; A61L 103/95

(52) U.S. Cl. CPC A61L 9/22; A61L 2/02; A61L 2103/95

(57) ABSTRACT

An apparatus for supplying surfaces, and in particular automotive surfaces, with ionized air, includes a supply connection for supplying compressed air to the apparatus, an ionization unit which is configured to ionize at least a portion of the air supplied via the supply connection, and a discharge device, which discharges the ionized air onto the surface. The ionization unit includes an energy conversion device that converts the kinetic energy of the air flow at least partially into electrical energy. The apparatus has a handle portion that serves to hold the apparatus, as well as a discharge portion for discharging the ionized air. The ionization unit is configured to ionize the air with a voltage that varies over time.

### CROSS REFERENCE TO RELATED APPLICATION

The present invention claims benefit of German Patent Application Serial no. 20 2025 102 452.7, filed May 5, 2025, the contents of which are incorporated herein by reference.

### BACKGROUND OF THE INVENTION

The present invention relates to an apparatus for ionizing air. In automotive engineering, it is known that surfaces to be painted, for example, are exposed to ionized air prior to painting. In this way, dust particles in particular can be removed from the aforementioned surfaces to be painted. Various application devices are known in the prior art for this purpose, with which ionized air can be applied to vehicle surfaces. Usually, for example, air guns are connected to high-pressure lines and thus discharge air. In addition, these guns are usually connected to a power supply that provides the air gun with voltage, and in particular high voltage, to generate the ionized air.

Furthermore, it is known from the applicant’s prior art that the surfaces to be treated are also electrostatically charged. This can occur, for example, through cleaning or wiping the surface. Here, the problem arises that the user must know how the surface is charged in order to use the correct (namely the same) charge when ionizing the air.

DE 10 2021 109 607 A1 discloses an apparatus for ionizing compressed air.

The present invention is therefore based on the objective of making the apparatuses described herein more user-friendly.

According to the invention, this is achieved by the subject matter of the independent claims. Advantageous embodiments and further developments are the subject of the dependent claims

Metal ionizers are known which have conductive tips supplied with high voltage that generate ions in the immediate vicinity through corona discharge and field emission. There are designs that continuously generate ions in an unregulated manner, and others in which the electric field is regulated by measuring and specifically adjusting the high voltage (regulated ionizer).

Regulated and unregulated ionizers are used both to build up defined charges and to dissipate unwanted charges (prevention of electrostatic charging and ESD).

In order to allow the ionizers to be positioned at a greater distance from the material to be discharged (up to approx. 2 m), they are also equipped with air nozzles or attached to existing nozzles. This blows the ions, along with the air, to the point of action (e.g., in printing presses).

Sawtooth ionizers are often designed as thin, comb-like strips. The term “sawtooth” here refers to the geometric design of the strips. They are powered byDC voltage or AC voltage. With AC voltage, all comb tips are connected together to the power source. With DC voltage, the metal tips arranged closetogether are alternately positively and negatively charged.

Consequently, ionization devices have become known in the prior art that operate on battery power, wherein this battery in turn generates a high voltage using corresponding electronic circuits, and this high voltage is used to ionize the air. On the one hand, this approach has the disadvantage that the batteries in the units are relatively heavy. On the other hand, such batteries must also be recharged regularly, which in turn leads to downtime and operational interruptions of the device.

DE 20 2018 101 625 discloses an ionization device that uses a generator driven by supplied compressed air to generate the voltage for ionizing the air. However, this ionization device is relatively cumbersome to use.

The objective is to create an apparatus that is more convenient to use compared to devices known from the prior art.

### SUMMARY OF THE INVENTION

An apparatus according to the invention for applying ionized air to surfaces, and in particular to automotive surfaces, comprises a supply connection for supplying compressed air to the apparatus. Furthermore, the apparatus comprises an ionization unit that is suitable and intended to ionize at least a portion of the air supplied via the supply connection. In addition, the apparatus comprises a discharge device that discharges ionized air, particularly onto a (particularly electrostatically charged) surface. However, the surfaces may also be walls, furniture surfaces, and generally surfaces of all kinds. Particularly preferred the surface is painted or a surface to be painted of a motor vehicle.

Furthermore, the ionization unit comprises an energy conversion device that converts the kinetic energy of the air flow at least partially into electrical energy.

According to the invention, the ionization unit is suitable and intended to ionize the air (flowing through it) using a voltage potential that varies over time and/or using a voltage that varies over time.

The surfaces to be treated are partially imparted with a predetermined charge. This can occur, for example, when wiping the surface with a cloth. The applicant has discovered that when using a varying voltage potential (and in particular a potential that is positive at times and negative at other times) for the air to be ionized or the ionizing voltage, it is possible to effectively expose the surface to be treated to ionized air (and, in particular, to discharge it) regardless of the charge of the surface.

Preferably, therefore, the apparatus causes the air to be ionized with alternating charge, and, for example, air that is positively ionized at times and negatively ionized at other times emerges from it.

Preferably, the generated ionized air is also ionized with a changing potential and/or a changing voltage, i.e., at times positive and at times negative. The applicant has surprisingly found that even the respective portion with the correct potential is sufficient for treating the respective surface.

In a preferred embodiment, the ionization unit is suitable and intended to ionize the air at least intermittently with a positive voltage and/or a positive voltage potential and at least intermittently with a negative voltage and/or a negative voltage potential. Preferably, the air is ionized with a voltage that, in particular, fluctuates and/or exposed to a voltage potential that fluctuates periodically, in particular.

Preferably, the apparatus and/or the ionization unit comprises an AC voltage generation device or an AC voltage generator, which is suitable and intended to generate an AC voltage that can be used to ionize the air.

Preferably, this AC voltage generator consists of several components, which are particularly preferred connected in series. This is explained below with reference to the figures.

Preferably, the AC voltage has a periodic waveform. Particularly preferred, this waveform is selected from a group of waveforms that includes sinusoidal or sinusoid-like waveforms, rectangular waveforms, sawtooth waveforms, triangular waveforms, combinations thereof, and the like.

Particularly preferred, the AC voltage generator or a transformer fed by the AC voltage generator generates a periodic AC voltage. This AC voltage may be an AC voltage with a temporal sinusoidal profile, a temporal triangular profile, a temporal rectangular profile, or the like.

Preferably, a temporal waveform is generated in which the output voltage remains above a predetermined threshold value over a longer period of time compared to a sinusoidal waveform. An example of such a temporal waveform is a temporal rectangular waveform. Such waveforms can be generated in particular by semiconductor circuits.

Preferably, this periodic AC voltage (in particular with respect to the highest voltage values) is greater than 100 V, preferably greater than 200 V, preferably greater than 400 kV, preferably greater than 600 V, preferably greater than 1 kV, preferably greater than 2 kV, preferably greater than 4 kV.

Preferably, this periodic AC voltage (in particular with respect to the highest voltage values) is less than 40 kV, preferably less than 30 kV, preferably less than 20 kV, preferably less than 15 kV.

These voltage values are not dangerous to a user holding the apparatus, as only very low currents flow. Nevertheless, the live components of the apparatus are preferably electrically isolated from the user and, in particular, from a hand of the user, which holds the apparatus.

In a further preferred embodiment, the energy conversion device supplies the ionization unit with electrical energy at least indirectly. In this design, an energy conversion device is therefore provided that converts at least a portion of the flow energy of the compressed air into electrical energy, and in particular into an AC voltage. This AC voltage, and in particular a voltage derived from this AC voltage, is in turn supplied to the ionization unit to thereby enable the ionization of the air.

In a further preferred embodiment, the AC voltage generator comprises an AC voltage conversion device that converts an input voltage (with a predetermined, essentially constant waveform and, preferably, an input AC voltage with a first predetermined temporal waveform) into an output AC voltage with a second predetermined waveform, wherein the second predetermined temporal waveform differs from the first predetermined temporal waveform.

Preferably, the first temporal waveform and the second temporal waveform differ from one another in at least one characteristic parameter. This refers in particular to a parameter that describes the temporal behavior of the voltage, such as a triangular profile or a rectangular profile in comparison to a sinusoidal profile.

In this design, the AC voltage conversion device causes a change in the temporal waveform of the AC voltage. For example, an input AC voltage which is generated by the generator device, and which has a sinusoidal waveform, can be converted into an output AC voltage with a triangular or rectangular waveform. It would also be conceivable for the temporal waveform of the output voltage to comprise a combination of different waveforms, such as initially a sinusoidal rise followed by a constant waveform starting at a certain threshold value.

However, it would also be possible for the input voltage to be a DC voltage, and for the AC voltage conversion device to generate an AC voltage with a predetermined temporal waveform from this DC voltage.

It would therefore also be possible for the generator device to generate a DC voltage.

In a further preferred embodiment, the AC voltage conversion device comprises a grounding. Thus, it would be possible, for example, for the output voltage to be output via a ground connection and a voltage control output.

Preferably, the AC voltage device is grounded via a pressure connection line.

In a further preferred embodiment, the AC voltage conversion device comprises a plurality of semiconductor elements.

Preferably, the AC voltage conversion device comprises a plurality of diodes. These can, for example, effect rectification of the input voltage.

Furthermore, the AC voltage conversion device preferably comprises at least one smoothing device for smoothing an input voltage. This smoothing device preferably comprises at least one capacitor.

In a further preferred embodiment, the AC voltage conversion device comprises at least one transistor and preferably at least two transistors. Preferably, these transistors serve to generate an AC voltage with a temporal profile that deviates from a sinusoidal profile.

In a further preferred embodiment, the AC voltage conversion device comprises a rectifier unit. This serves in particular to rectify the input voltage.

The applicant has discovered that a sinusoidal voltage is less suitable for ionization, as only certain portions of the temporal waveform cause ionization. The triangular or rectangular voltages described here have proven to be better suited for ionizing the air with different polarities.

Preferably, an ionization element of the ionization unit is designed such that compressed air flows past two electrode regions or between two electrode regions.

Preferably, an ionization element of the ionization unit is designed such that the flow section along which the compressed air flows during ionization is shorter than 20 cm, preferably shorter than 15 cm, preferably shorter than 10 cm, preferably shorter than 8 cm, preferably shorter than 6 cm, preferably shorter than 4 cm, preferably shorter than 3 cm, and particularly preferred shorter than 2 cm.

Preferably, an ionization element of the ionization unit is designed such that the flow section along which the compressed air flows during ionization is longer than 0.05 cm, preferably longer than 0.1 cm, preferably longer than 0.2 cm, and particularly preferred longer than 0.4 cm.

In a further advantageous embodiment, the AC voltage conversion device is supplied (in particular directly) by the energy conversion device. In a further preferred embodiment, an output signal and/or an output voltage of the energy conversion device is supplied at least indirectly to the AC voltage conversion device as an input signal.

In a further preferred embodiment, the AC voltage conversion device comprises a transformer device which transforms, and in particular steps up, an intermediate voltage generated by a voltage generation unit of the AC voltage conversion device into an output voltage of the AC voltage conversion device.

In a further advantageous embodiment, the apparatus comprises a charge generation device that is suitable and intended to electrically charge the surface to be treated and, in particular, to charge it to a preferably constant potential. This device may, for example, be a textile element such as a cloth, which generates the charge when it is moved over the surface of the vehicle. In this context, it is not readily predictable with which polarity the surface will be charged.

However, exposure to both positively and negatively ionized air preferably causes a discharge of the surface.

In a further preferred embodiment, the AC voltage generator generates a periodic AC voltage, and this periodic AC voltage has a frequency that is higher than 100 Hz, preferably higher than 200 Hz, preferably higher than 400 Hz, preferably higher than 800 Hz, preferably higher than 1000 Hz, and particularly preferred higher than 2000 Hz.

Preferably, this AC voltage has a frequency lower than 100 kHz, preferably lower than 50 kHz, preferably lower than 40 kHz, preferably lower than 30 kHz, and preferably lower than 20 kHz.

Advantageously, the apparatus comprises a handle portion for holding the apparatus and a discharge portion for discharging the ionized air, wherein, particularly preferred, the energy conversion device is integrated at least in part into the handle portion. Preferably, a generator device of the energy conversion device is arranged in the handle portion.

Particularly preferred, the energy conversion device directly generates the AC voltage used to ionize the air. Preferably, the energy conversion device is therefore well-suited for generating an AC voltage.

In this way, a more convenient arrangement of the apparatus can be achieved, since comparatively heavy components are integrated into the handle portion. Furthermore, the airflow of the supplied compressed air can also be utilized in a particularly advantageous manner, since the energy conversion takes place very close to the compressed air supply.

Preferably, the handle portion and the discharging portion extend in different directions. In this context, these directions are particularly preferred such that an angle is formed between them that is greater than 50°, preferably greater than 60°, preferably greater than 70°, and particularly preferred greater than 80°. Preferably, this angle is less than 150°, preferably less than 130°, preferably less than 120°, preferably less than 110°, and particularly preferred less than 100°.

In an advantageous embodiment, the energy conversion device comprises a generator device that converts the rotational movement of a rotor relative to a stator into electrical energy, wherein the generator device is particularly preferred designed as an external rotor machine. In a particularly preferred embodiment, the energy conversion device and/or the generator device is integrated into the handle portion.

By designing the generator device as an external rotor machine the ergonomics and comfort of the apparatus can also be improved, since this design allows the generator device to be made smaller and the airflow channels inside the housing to be arranged in a more space-saving manner.

This means that, preferably, the stator of the generator device is arranged on the inside and the rotor, which rotates relative to the stator, is arranged on the outside. An external rotor is a type of rotating electrical machine in which the stationary part (stator) of the machine is located inside and is enclosed by the moving part (rotor). Depending on the operating point and design, the machine can be used as an electric generator or as an electric motor.

The advantage of designing the generator device as an external rotor machine lies in the fact that the airflow can be utilized more effectively to generate the rotational movement of the rotor. In this way, the generator device can also be designed to be smaller and thus lighter.

A further apparatus according to the invention for applying ionized air to surfaces, and in particular to automotive surfaces, comprises a supply connection for supplying compressed air to the apparatus. Furthermore, the apparatus comprises an ionization unit that is suitable and intended to ionize at least a portion of the air supplied via the supply connection. In addition, the apparatus comprises a discharge device that discharges ionized air, and in particular onto a surface.

Furthermore, the ionization unit comprises an energy conversion device that converts the kinetic energy of the air flow at least partially into electrical energy.

According to the invention, the energy conversion device comprises a generator device that converts the rotational movement of a rotor relative to a stator into electrical energy, wherein the energy conversion device is preferably designed as an external rotor machine. Preferably, the energy conversion device generates an AC voltage.

In an advantageous embodiment, the rotor of the generator device comprises air intake devices, such as air vanes, on its outer circumference, which can be moved by the passing air. Preferably, a channel runs at least in sections around the generator device, through which the supplied compressed air flows.

In a further preferred embodiment, an axis of rotation of the rotor of the generator device comprises a component in a direction extending in the direction of exit of the air leaving the apparatus.

Preferably, the direction of exit of the (ionized) air leaving the apparatus and the direction of supply define a plane, and the axis of rotation of the rotor also extends in this plane or in a plane parallel thereto. Preferably, the axis of rotation of the rotor extends at an angle relative to a main flow direction of the air exiting the apparatus that is less than 40°, preferably less than 30°, preferably less than 25°, preferably less than 20°, preferably less than 15°, and preferably less than 10°.

In a further advantageous embodiment, the generator device is at least partially, and particularly preferred completely, integrated into the handle portion of the apparatus.

In a further advantageous embodiment, the apparatus comprises at least two housing units, wherein the energy conversion device is arranged in the first housing unit and this first housing unit is arranged at least partially within the second housing unit. It should be noted that this variant also offers advantages independent of the design described above, in particular with regard to the electrical insulation of the apparatus. However, specific advantages may also arise from the combination of both embodiments, as this achieves better electrical insulation, in particular with respect to the generator device, which is preferably located in the handle portion.

A further apparatus according to the invention for applying ionized air to surfaces, and in particular to automotive surfaces, comprises a supply connection for supplying compressed air to the apparatus. Furthermore, the apparatus comprises an ionization unit that is suitable and intended to ionize at least a portion of the air supplied via the supply connection. In addition, the apparatus comprises a discharge device that discharges ionized air and in particular onto a surface. Furthermore, the ionization unit comprises an energy conversion device that converts the kinetic energy of the air flow at least partially into electrical energy.

According to the invention, the apparatus comprises at least two housing units, wherein the energy conversion device is arranged in the first housing unit and this first housing unit is arranged at least partially within the second housing unit. In particular, the energy conversion device generates an AC voltage.

In a further preferred embodiment, the second housing unit is designed as a enclosing housing. Preferably, the second housing part is made of an electrically insulating material, and in particular of plastic. Particularly preferred the entire outer surface of the housing is made of plastic. In this way, it can be prevented that a user comes into contact with the voltage used for ionization.

Preferably, the second housing part is formed at least in the handle portion of the apparatus. However, it would also be conceivable for the second housing part to substantially completely enclose the first housing part.

In a further preferred embodiment, the second housing part comprises multiple parts and/or at least two parts. In this case, it is possible for these two housing parts to be fastened together by means of a clip mechanism.

In a further preferred embodiment, the second housing unit comprises at least two housing parts that can be connected to one another. Preferably, these two housing parts are held together (at least in part) by a compressed air connection.

In a further advantageous embodiment, the apparatus comprises a sensor device for detecting air pressure or air flow. This may, for example, be a flow meter or a pressure gauge. However, it would also be possible for the sensor device to be a current or voltage measuring device that measures a voltage or current generated by the energy conversion device, since this voltage or current serves as a measure of the compressed air pressure or its flow velocity.

In a further preferred embodiment, the apparatus comprises a warning device that emits a warning signal when the actual pressure deviates from a target pressure, in particular when the air pressure is too high or too low. In particular, the warning device is an optical or visually perceptible warning device. This may, for example, be arranged in a transition area between the handle portion and the discharge portion so as to be readily visible to a user at all times.

It is possible that this warning device does not have its own power source but is supplied with energy by the energy conversion device.

It is therefore proposed that, preferably, the airflow itself be used to achieve ionization. In particular, therefore, an energy conversion unit is provided that enables the conversion of the kinetic energy of the airflow into electrical energy.

Instead of air, another gaseous medium could also be used, such as sterilized air or a sterilizing gas.

Preferably, the discharge device which is, in particular, an air discharge device is arranged on the ionization unit. This allows the discharge device to be screwed onto the ionization unit. The air discharge device may also preferably be suitable and intended to generate an electric field for ionization.

In a preferred embodiment, the apparatus comprises a lighting device that emits a visually perceptible signal and that, in particular, illuminates when the energy conversion device is supplied with energy by the air flow.

In a preferred embodiment, the apparatus may comprise an air filter unit to filter the supplied air. Furthermore, the apparatus may comprise a drying unit for drying the air flowing through it. However, it would also be possible for the dried and/or sterilized or purified air to already be supplied via the supply connection.

In a further advantageous embodiment, the energy conversion device comprises a piezoelectric element and/or a dynamo device. In this way, it can be achieved that the air flow, for example, indirectly moves a piezoelectric element and thus generates voltages, or that the air flow drives a dynamo unit, which in turn generates an AC voltage, and in particular an AC voltage.

In the aforementioned embodiments, the apparatus can therefore be powered solely by the airflow, wherein said airflow also provides the energy required for ionization.

In a further advantageous embodiment, the energy conversion device comprises a rotatable turbine element that can be set in rotation in particular by the said airflow. For example, this may be a type of paddle wheel that is set in rotation by the airflow. This rotation, in turn, causes the generation of electrical energy by the energy conversion device. As mentioned above, this turbine wheel is preferably a component of an external rotor machine.

In a further advantageous embodiment, an air duct is connected to the turbine element, which ultimately conveys the air to the discharge device.

In a further advantageous embodiment, the ionization unit comprises a high-voltage generation unit. Preferably, this high-voltage generation unit generates an AC voltage. For example, the high-voltage generation unit may be a transformer that transforms the AC voltage supplied by the energy conversion device into a high voltage. Preferably, this high-voltage generation unit is arranged outside the handle portion and, particularly preferred, in the discharge portion. Preferably, this high-voltage generation unit is suitable and intended to generate an AC voltage.

This high voltage can then be applied to a region of the discharge device. For example, an electrode or electrical cable may extend from the voltage generation unit to a nozzle cap, and a second cable may extend to a needle located centrally relative to this nozzle cap, so that an electric field can be established between these two elements, which ultimately causes the ionization of the air.

In addition, however, it would also be possible for the apparatus to comprise a rectifier unit that is suitable and intended to convert the AC voltage generated by the high-voltage generation unit into a DC voltage.

In a further preferred embodiment, the apparatus comprises at least one capacitor and/or at least one diode. These elements may, for example, serve to rectify or smooth the voltage output by the high-voltage generation unit.

In a further advantageous embodiment, the high-voltage generation unit comprises a transformer unit, as mentioned above.

In a further advantageous embodiment, the high-voltage generation unit is electrically supplied to and/or connected to the energy conversion unit.

In a further advantageous embodiment, the discharge device comprises a nozzle element via which an air flow can be applied, in particular to the surface to be treated.

Preferably, as mentioned above, this nozzle element may be one of the two electrical electrodes that are electrically connected to the AC voltage or high-voltage generation unit. In a further advantageous embodiment, the apparatus comprises a flow distribution device that divides a flowing air stream preferably into multiple openings. These multiple openings may be arranged in a circumferential direction around a centrally located electrode. Particularly preferred, these openings can be distributed regularly and/or irregularly.

In a further preferred embodiment, the apparatus comprises a grounding device for electrically grounding the apparatus during operation. Particularly preferred, one of the two electrodes is in electrically conductive contact with this grounding device. Preferably, therefore, at least one of the two electrodes is electrically grounded.

Preferably electrical components of the AC voltage conversion device are grounded via the grounding device.

In a further preferred embodiment, the discharge device comprises an electrically conductive connection means for connecting the discharge device to a grounding line. For example, a fastening means by which a pressure hose is attached to the discharge device or to the apparatus may also serve to establish an electrically conductive connection for the purpose of grounding.

In a further preferred embodiment, at least one parameter characteristic of the voltage used by the ionization unit for ionization is adjustable. In this way, a user can adjust the voltage, for example, if they know how the surface is charged or depending on the surfaces to be treated.

Preferably, this parameter is selected from a group of parameters that includes a frequency of the voltage, a maximum positive voltage, a maximum negative voltage, a temporal waveform of the voltage, a symmetry of the voltage with respect to a predetermined potential, and the like.

In a further advantageous embodiment, air can be supplied via the supply connection at a pressure greater than 0.1 bar, preferably greater than 0.5 bar, preferably greater than 1 bar, preferably greater than 2 bar.

Furthermore, air is preferably supplied via the supply connection at a pressure that is less than 15 bar, preferably less than 12 bar, preferably less than 10 bar, preferably less than 9 bar, and particularly preferred less than 8 bar.

These pressures have proven to be particularly advantageous for ensuring a sufficient exposure to air to the respective surfaces even during subsequent ionization.

In a further advantageous embodiment, the apparatus comprises a pressure control device that regulates a pressure of the air or the air flow reaching the energy conversion device. In a preferred embodiment, this pressure control device is arranged at the air connection. Particularly preferred the pressure control device is arranged in the direction of the airflow, upstream relative to the connection. In this way, a particularly stable design of the pressure control device is possible.

With the aid of the pressure control device, the respective desired pressure can be set even before reaching the energy conversion device.

In a further advantageous embodiment, the apparatus is a hand-held or portable device. For example, the apparatus may be designed as a spray gun that can be held by a user to apply ionized air to the surface of the motor vehicles. In a further preferred embodiment, the supply connection comprises a metal thread via which an (air) pressure source can be connected.

In a further preferred embodiment, the apparatus comprises a lighting device for illuminating the surface to be treated, particularly an automotive surface. This lighting device may, for example, be arranged at the head of the apparatus and directed toward the surface to be treated. In a preferred embodiment, the lighting device comprises at least one and preferably several light sources, in particular light-emitting diodes. These light sources may be arranged around an outlet opening from which the ionized air exits. In this way, the ionization surface may be easier to see.

In a preferred embodiment, the lighting device comprises at least one white light diode.

In a preferred embodiment, the lighting device emits light in the form of a light cone suitable for illuminating an area ranging from 1 cm² to 1000 cm². Preferably, the size of this light cone is adjustable.

In a preferred embodiment, the lighting device comprises an LED and/or a laser and/or a laser diode. In a further preferred embodiment, the lighting device comprises an optically refractive element and, in particular, a lens assembly.

In a preferred embodiment, the focal length of this lens assembly is selected such that it spreads the light emerging from the lighting device in approximately the same manner as the discharged air stream (which is discharged through the nozzle).

In this way, it is possible for a light spot to be approximately as large as the “spray cone” of the discharged air, regardless of its distance.

In a further preferred embodiment, the lighting device is arranged and/or oriented such that it produces a light spot on the workpiece to be treated that is at least partially congruent with the area onto which the ionized air is directed. Preferably, the light spot emitted by the lighting device onto the surface to be treated is smaller than the area of ionized air produced by the apparatus. This ensures that a user operating the apparatus and orienting themselves by the light spot will ionize all desired surface areas at any rate.

Preferably, the lighting device can be triggered and activated simultaneously with the spraying process. For example, a push button may be provided that is activated at the same time or with a slight time delay.

Preferably, the lighting device is powered by electrical energy generated by the generator from the air flow. However, it would also be possible for the apparatus to comprise a rechargeable energy storage device, such as a battery or a capacitor, which is preferably powered by the energy generated by the generator.

In a further preferred embodiment, the lighting device is suitable and designed to form a light cone and/or a light spot on the surface to be treated, which has a cross-section deviating from a circular cross-section. In particular, this is an elliptical cross-section.

Particularly preferred, the lighting device comprises at least one optical element suitable for producing a light cone and/or an illumination of the surface to be treated with an elliptical cross-section. Particularly preferred, the optical element is a cylindrical lens.

The generation of an elliptical light spot enables a more precise adaptation to the profile of the airflow discharged by the apparatus to the surface.

Particularly preferred, the ellipticity of the illumination emitted by the lighting device is adjustable. This can be achieved, for example, by adjusting the distance between the light source or light sources and an optical element.

In a further preferred embodiment, the apparatus comprises a distance-detection device that detects a distance between the apparatus and a wall to be treated , and in particular detects it without contact. This may, for example, be an infrared distance-detection device.

In a further preferred embodiment, the apparatus comprises a control device that controls the lighting device in reaction to a distance determined by the distance detection device. For example, it is possible for the lighting to be switched off if a distance between the apparatus and the surface to be treated is too great or exceeds a predetermined threshold value.

In a further preferred embodiment, the distance detection device is also powered by electrical energy obtained from the airflow.

In a further preferred embodiment, the apparatus comprises a time-recording device. In this context, the time-recording device may record temporal quantities, such as the time and/or duration of a work operation. Additionally, the time-recording device can record and/or count operating hours.

In a further preferred embodiment, the apparatus comprises a storage device in which time values and/or durations can be stored. In this way, work operations can be logged. Furthermore, the apparatus preferably comprises a communication interface for communicating with, for example, a computer. This communication interface may operate via a cable or wirelessly.

In a further advantageous embodiment, the piezo element or a carrier supporting or bearing the piezo element protrudes, at least in sections, into a flow of the air. In this way, the piezo element can be directly excited into motion by the air flow and thus generate a voltage.

In a further advantageous embodiment, the supply connection allows for a flow cross-section of the air entering the apparatus that is greater than 0.05 mm, preferably greater than 0.1 mm, particularly preferred greater than 0.15 mm, and particularly preferred greater than 0.2 mm.

Preferably, the supply connection allows for a flow cross-section of the air entering the device that is less than 20 mm, preferably less than 18 mm, and preferably less than 15 mm, and particularly preferred less than 10 mm.

In a further advantageous embodiment, the apparatus comprises a valve device for controlling the air flow from the supply connection to the discharge device. Preferably, this valve device is arranged in the direction of the air flow, upstream of the ionization unit. Preferably, this valve device is at least indirectly manually operable.

Preferably, this valve device is designed such that it can be actuated by a switching element, such as a pivotable handle of the switch or the like. Particularly preferred a return device and/or a spring device is provided, which urges this valve device into a closed state, so that the valve device blocks an air flow without actuation of the apparatus.

In a further advantageous embodiment, the apparatus comprises a trigger element for actuating this valve device. Thus, this trigger element may be designed such that the air pressure of the ionized air reaching the discharge device can also be controlled via the trigger element.

In another advantageous embodiment, the apparatus comprises a housing in which an air duct is arranged at least in sections. This air duct may extend at least in sections from the supply connection to the discharge device, with the energy conversion unit and/or the turbine wheel being arranged between the supply connection and the discharge element or discharge device.

In a further advantageous embodiment, the apparatus comprises a conduit for conducting the air, and this conduit changes the flow direction of the air at least once. For example, a conduit device may change the flow direction of the air by a predetermined angle, in particular an angle between 10° and 170°, such as 90°.

In a further preferred embodiment, the apparatus is suitable and intended to at least partially discharge a statically charged surface and/or to eliminate the charge by the exposure.

In a further preferred embodiment, the apparatus comprises at least one ionization element formed with a tip and, preferably, at least two ionization elements formed with a tip, which ionize air flowing past these ionization elements.

The present invention further relates to a method for treating and, in particular, for painting surfaces of motor vehicles, wherein an automotive surface is exposed to ionized air.

According to the invention, the ionized air is generated by an apparatus of the type described above. Particularly preferred, a flowing air generates a voltage, which in turn is used to ionize the air.

The present invention further relates to a method for sterilizing and/or cleaning surfaces, wherein a surface to be sterilized or cleaned is exposed to a gaseous medium, and in particular to air.

According to the invention, the ionized air is generated by means of a apparatus of the type described above.

In a preferred embodiment, dried air and/or purified air is supplied to the apparatus. Particularly preferred, as mentioned above, air is supplied at pressures of at least 0.5 bar, preferably at least 1 bar, and particularly preferred at least 2 bar. Particularly preferred, air is supplied at a pressure lower than 15 bar, preferably lower than 12 bar, preferably lower than 10 bar, and particularly preferred lower than 8 bar.

In a further preferred method, the air is ionized by a discharge device that also discharges the air. Advantageously, a generated AC voltage is converted and, in particular, transformed. In a further preferred method, the aforementioned apparatus is held by hand. Particularly preferred, the air is applied at an angle between 5° and 50° relative to the surface or the plane of the surface.

In a preferred method, a charge on the surface, and in particular a static charge on the surface, is reduced by the aforementioned apparatus and preferably substantially removed.

In a further preferred method, the said surface is painted after being exposed to ionized air. Particularly preferred, this painting takes place within 20 minutes, preferably within 15 minutes, preferably within ten minutes, preferably within eight minutes, preferably within 5 minutes, and particularly preferably immediately after the surface has been exposed to ionized air.

In a further preferred method, an AC voltage is used to ionize the air. Particularly preferred, the AC voltage has a time wave form selected from a group of time wave forms comprising triangular, rectangular, and sinusoidal waveforms .

Particularly preferred an apparatus of the type described above is used

Particularly preferred, the apparatus described above is arranged such that there is a distance of more than 10 cm between the air discharge of the air of the apparatus and the surface to be treated.

Particularly preferred, the surfaces to be treated are surfaces of motor vehicles. In particular, these are surfaces of motor vehicles to be painted. Particularly preferred, the surfaces are painted after being exposed to ionized air.

### BRIEF DESCRIPTION OF THE DRAWINGS

Further advantages and embodiments are apparent from the accompanying drawings:

### These show

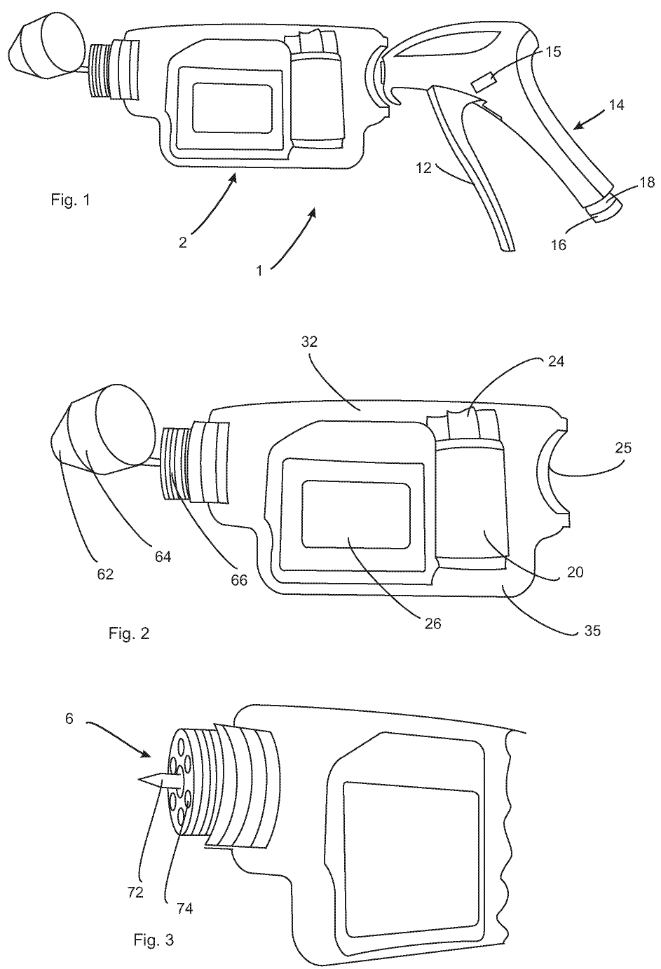

FIG. 1 A schematic representation of an apparatus according to the invention;

FIG. 2 A representation of the ionization unit of the apparatus shown in FIG. 1;

FIG. 3 A detailed view of the ionization unit shown in FIG. 2;

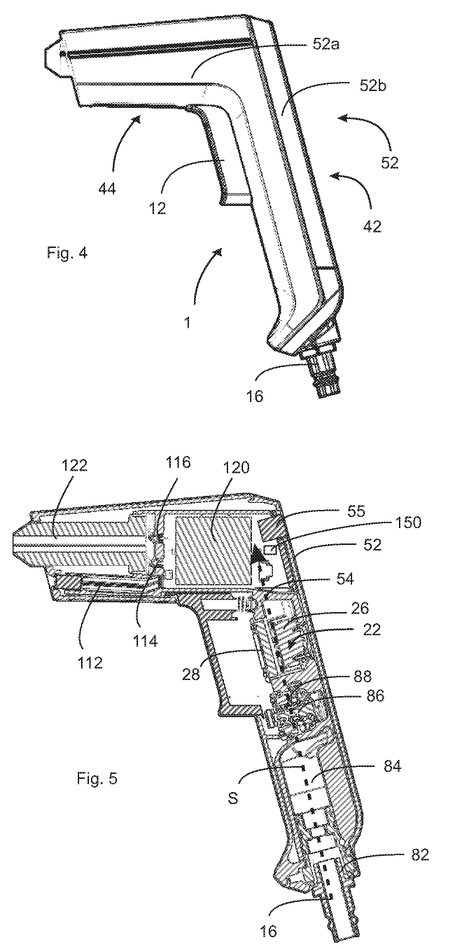

FIG. 4 A representation of a further design of the apparatus according to the invention;

FIG. 5 a sectional representation of the apparatus shown in FIG. 4;

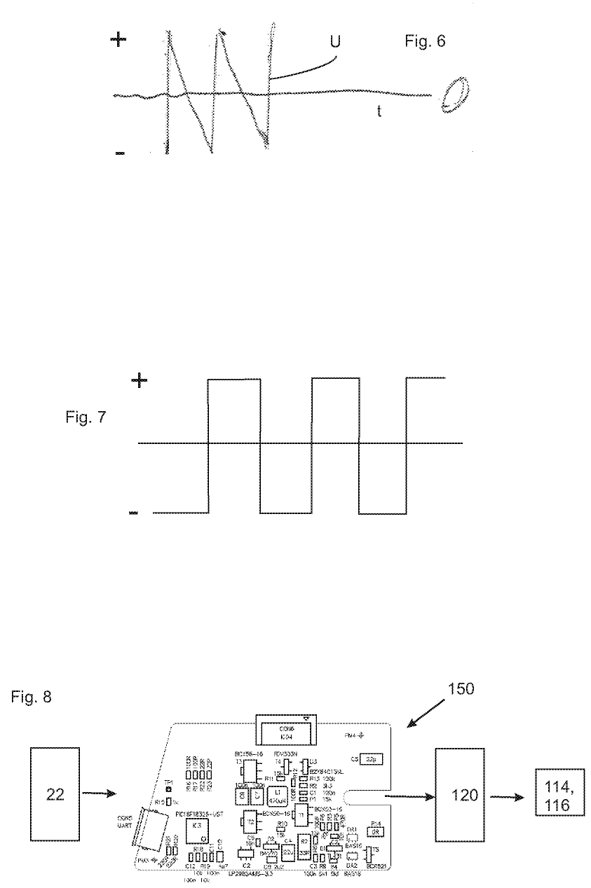

FIG. 6 a voltage waveform of an output voltage for ionization in a first embodiment;

FIG. 7 a voltage waveform of an output voltage for ionization in a second embodiment;

FIG. 8 a schematic representation illustrating voltage generation;

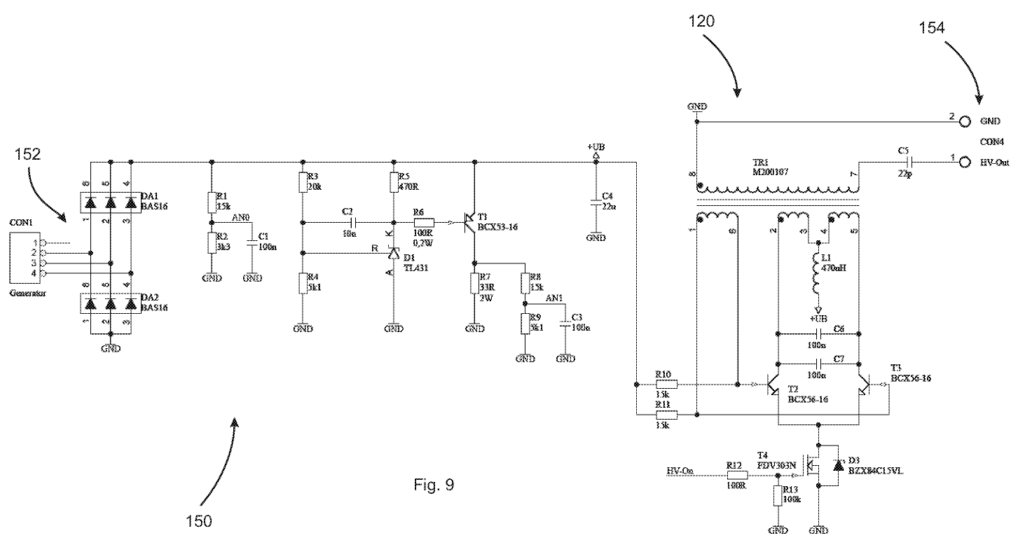

FIG. 9 a circuit diagram of an AC voltage conversion device.

### DETAILED DESCRIPTION OF THE INVENTION

FIG. 1 shows a schematic representation of an apparatus 1 according to the invention for ionizing air. This apparatus comprises a supply connection 16 through which compressed air can be supplied to the apparatus. Reference number 18 denotes a pressure control unit, which is in particular manually adjustable to change the air pressure.

Reference number 14 denotes a base body, and reference number 12 denotes an actuating element, such as a push button or a switch, with which a user can control the compressed air output. Preferably, a valve device 15 may be located inside the base body, which can in particular also be actuated by the push button or switch 12.

In this way, the compressed air enters the ionization unit, designated in its entirety by the reference number 2.

FIG. 2 shows a more detailed view of the ionization unit 2. This unit comprises a supply connection 25. Compressed air is supplied to the ionization unit via this connection. The ionization unit 2 may in its entirety be arranged on the base body 14, in particular via a screw connection.

The reference number 20 denotes an energy conversion device. This energy conversion device comprises a turbine wheel 24, which can be driven by the air flow of the compressed air. In the figure, this turbine wheel would rotate about a vertical axis. The energy conversion device may comprise a piezoelectric element that converts this mechanical movement of the turbine element or the mechanical movement generated by the turbine element into an AC electrical voltage. However, the energy conversion device 20 could also include a dynamo unit that generates AC voltage.

The AC voltage generated by the energy conversion device 20 is output to a high-voltage generation unit 26. This high-voltage generation unit 26 may, in particular, comprise a transformer unit that transforms the AC voltage.

Reference number 32 denotes a channel or a conduit section that serves to lead or conduct the compressed air. From this channel 32, the air reaches, in particular, the discharge device designated in its entirety by 6. In this discharge device, reference number 66 denotes a thread onto which a discharge nozzle 62 can be screwed. This nozzle 62 is preferably made of an electrically conductive material, in particular a metal. Reference number 64 denotes a base body of the discharge device.

FIG. 3 shows a representation of the discharge device 6 of the apparatus according to the invention. It can be seen that a plurality of openings 74 are provided here, which are arranged in a circumferential direction around a needle electrode 72. The compressed air can be discharged through these openings 74 and is thereby ionized between two electrodes. One of these electrodes is formed by the surface 62 or 64, and the other electrode, as mentioned, by the needle 72. Thus, as mentioned above, the air can be ionized by the electric field between this needle tip 72 and the areas 62 and 64.

FIG. 4 shows a further design of an apparatus 1 according to the invention. This apparatus comprises a housing or an outer housing part 52. The apparatus further comprises a handle portion 42 that can be held by a user, as well as a discharge portion through which the gaseous medium can be discharged.

Reference number 16 denotes the supply connection through which a gaseous medium, and in particular (compressed) air, can be supplied to the apparatus. Preferably, the outer housing part 52 is formed by two parts and comprises a housing body 52a and a housing cover 52b. These may preferably be connected to and/or via the supply connection or an element of this supply connection.

FIG. 5 shows a sectional view of the apparatus shown in FIG. 4. Compressed air is preferably supplied to the apparatus 1 via the supply connection. The supply connection is preferably arranged on the handle portion. Reference number 82 denotes a coupling or an adapter to which the air supply can be connected.

Reference number 84 denotes an optional air reservoir to which the compressed air is supplied. In addition, the apparatus may preferably also comprise (not shown) filter devices for filtering the incoming air, such as HEPA filters.

Reference number 86 denotes a switchable valve, which is preferably forced into a closed position by a spring and which can preferably be actuated and thus opened by means of the trigger element shown in FIG. 4.

In this way, the compressed air reaches the generator device designated by reference number 22. This generator device 22 comprises a stator 28 and a rotor 22 rotatable relative to said stator. This rotor is driven to rotate by the compressed air.

The reference symbol S denotes the main flow direction of the air flowing within the housing or the handle portion 42. In the design shown in FIG. 5, the axis of rotation of the rotor is essentially perpendicular to the main flow direction S.

Thus, the pneumatic pressure drives the generator device, whereby the design of the generator device as an external rotor machine provides improved passage for the compressed air.

The power generated in the generator device is supplied to an input power for the apparatus and/or an input circuit board and/or a transformer 120. When the input power exceeds a certain voltage, this voltage is applied to the two pins 114 and 116 (or to one of the two pins), and these pins ionize the passing air. Preferably, an AC voltage can be applied to pins 114 and 116. It is also and preferably possible for one of the two pins to be grounded.

Reference number 150 denotes an AC voltage conversion device that generates a predetermined (temporal) waveform of the output voltage, such as a triangular waveform. Preferably, this predetermined temporal waveform is variable.

Preferably, for example, the magnitude of the output voltage is also variable.

In this context, the transformer is preferably arranged in the discharge portion 44 of apparatus 1. Reference number 122 denotes the actual nozzle through which the compressed air is discharged to the outside.

Reference number 112 denotes a lighting device, which is preferably also electrically powered by the transformer 120 and thus also by the generator device 22. This lighting device preferably comprises a lens and, particularly preferred, a cylindrical lens.

Preferably, a direction of radiation of the lighting device 112 is slightly inclined relative to the direction of airflow within the nozzle 122, such that, at a predetermined distance between the apparatus and a surface to be treated, the light cone of the radiation emitted by the lighting device and the cone of the outgoing air coincide with good approximation.

For example, the apparatus may be calibrated or adjusted to a specific distance between the apparatus and the surface to be treated, such as a spray distance between 10 cm and 20 cm.

In addition, it would also be possible to alter the direction of radiation in which the lighting device 112 emits its radiation, so that the distances at which the light cone of the radiation emitted by the lighting device and the “spray cone” of the air discharged by the nozzle device essentially coincide. In this way, the lighting device can indicate to the user the surface area currently being exposed to the ionized air.

The reference number 52 denotes the outer housing of the apparatus, within which an inner housing 54 is preferably arranged, which particularly preferred also carries at least one component, preferably several components of the device (such as the generator device or the transformer). In this way, electrical insulation can also be achieved in a particularly advantageous way. Preferably, the two pins that cause ionization are also arranged within this inner housing.

Reference number 55 denotes a warning device such as a warning light. This may preferably be designed as a multicolored lighting that can provide indications to the user through different light outputs. For example, a flashing of this warning device may indicate that the pressure at connection 16 is too low, a constant, for example green, light may indicate that the pressure is within a desired range, and a red light could indicate that the pressure is too high.

Furthermore, the apparatus may comprise a measuring device that records at least one measured value characteristic of the applied air pressure. For example, the applied pressure could directly be determined via a pressure measuring device. Also, a flow meter could be used to measure the flow velocity of the passing air, or the air pressure or flow velocity could be inferred based on the generated voltages and/or currents.

Preferably, the apparatus comprises a processor device (not shown) that emits a signal to the warning device based on the measured value provided by the measuring device.

FIG. 6 shows a voltage waveform of the voltage applied to the ionization unit and/or its electrodes. It can be seen that this voltage follows a periodic waveform and, in the particularly preferred design shown in FIG. 6, takes on a sawtooth-like waveform.

In this case, the voltage rises abruptly from an initially negative potential to a positive potential and then decreases, in particular with a constant gradient, back to the negative potential.

Preferably, the positive (voltage) potential relative to 0 and/or relative to the grounding and the negative (voltage) potential correspond with each other or deviate from each other by no more than 20% and preferably by no more than 10%.

FIG. 7 shows another possible voltage waveform, here in the form of a rectangular profile

FIG. 8 shows a schematic representation of voltage generation. The energy conversion device 22 generates an (AC ) voltage from the compressed air. This AC voltage is supplied as an input signal or input voltage to an AC voltage conversion device 150.

This AC voltage conversion device generates an output signal in the form of a triangular voltage or with a triangular waveform. This triangular voltage is preferably transformed by a transformer device, which, however, preferably forms a component of the AC voltage conversion device.

The AC voltage generated in this manner is supplied to the electrodes 114, 116 for ionizing the compressed air.

FIG. 9 shows a schematic diagram of an AC voltage conversion device 150. This device has a voltage input 152 through which the input voltage, in particular from the energy conversion device, is supplied to the AC voltage conversion device.

Reference number 154 denotes an output via which the AC voltage generated by the AC voltage conversion device is output, in particular to the electrode(s).

Preferably, the input voltage is rectified and smoothed. Preferably, a voltage with a desired temporal profile is generated by means of two transistors. This is transformed by means of a transformer device and output to the output 154.

It should be noted that all features described with reference to the method are also disclosed in a corresponding manner for the apparatus, which in particular means that the corresponding apparatus comprises devices suitable and intended for carrying out the respective methods. Furthermore, features described with reference to the apparatus are also applicable to the method or the methods in a corresponding manner. This means that the methods are carried out using the corresponding apparatus features.

The applicant reserves the right to claim all features disclosed in the application documents as essential to the invention, provided they are novel, individually or in combination, relative to the prior art. It is further noted that the individual figures also describe features which may be advantageous in and of themselves. The skilled person immediately recognizes that a specific feature described in a figure may be advantageous even without adopting further features from that figure. Furthermore, the skilled person recognizes that advantages may also result from a combination of several features shown in individual or different figures.

1. An apparatus for supplying surfaces, and in particular automotive surfaces, with ionized air, comprising a supply connection for supplying compressed air to the apparatus, an ionization unit which is configured to ionize at least a portion of the air supplied via the supply connection, and a discharge device, which discharges the ionized air onto the surface, wherein the ionization unit comprises an energy conversion device that converts the kinetic energy of the air flow at least partially into electrical energy, wherein the apparatus comprises a handle portion that serves to hold the apparatus, as well as a discharge portion for discharging the ionized air,
wherein
the ionization unit is configured to ionize the air with a voltage that varies over time.

2. The apparatus according to claim 1,
wherein
the ionization unit is configured to ionize the air at least intermittently with a positive voltage and at least intermittently with a negative voltage.

3. An apparatus according to claim 1,
wherein
the ionization unit comprises an AC voltage generator that is configured to generate an AC voltage.

4. The apparatus according to claim 1,
wherein
the AC voltage generator is configured to generate a periodic AC voltage, and the periodic AC voltage has a sinusoidal waveform or a sawtooth waveform or a rectangular waveform or combinations thereof.

5. The apparatus according to claim 1,
wherein
the energy conversion device is integrated at least in part into the handle portion.

6. The apparatus according to claim 1,
wherein
the AC voltage generator is configured to generate a periodic AC voltage, and this periodic AC voltage has a frequency that is higher than 100 Hz, preferably higher than 200 Hz, preferably higher than 400 Hz, preferably higher than 800 Hz and preferably higher than 1000 Hz and particularly preferred higher than 2000 Hz and/or preferably lower than 50 kHz, preferably lower than 40 kHz, preferably lower than 30 kHz, preferably lower than 20 kHz.

7. The apparatus according to claim 1,
where
the AC voltage generator or a transformer supplied by the AC voltage generator is configured to generate a periodic AC voltage, and this periodic AC voltage is greater than 100 V, preferably greater than 200 V, preferably greater than 400 V, preferably greater than 600 V, preferably greater than 1 kV, preferably greater than 2 kV, preferably greater than 4 kV, and/or this periodic AC voltage is less than 40 kV, preferably less than 30 kV, preferably less than 20 kV, preferably less than 15 kV.

8. The apparatus according to claim 1,
wherein
the energy conversion device is configured to supply the ionization unit with electrical energy at least indirectly.

9. The apparatus according to claim 1,
wherein
the AC voltage generator comprises an AC voltage conversion device which is configured to convert an input voltage, and in particular an input AC voltage with a first predetermined temporal waveform, into an AC output voltage with a second predetermined waveform, wherein the second predetermined temporal waveform differs from the first predetermined temporal waveform .

10. The apparatus according to claim 9,
wherein
the AC voltage generator is supplied by the energy conversion device and/or an output signal and/or an output voltage of the energy conversion device is supplied at least indirectly to the AC voltage generator as an input signal.

11. The apparatus according to claim 8,
wherein
the AC output voltage is supplied to an ionization element and/or at least one electrode, which ionizes the air.

12. The apparatus according to claim 1,
wherein
the apparatus comprises a grounding device for electrically grounding the apparatus during operation.

13. The apparatus according to claim 1,
wherein
the discharge device comprises an electrically conductive connection for connecting the discharge device to a ground line.

14. The apparatus according to claim 1,
wherein
the apparatus comprises a connection line by which compressed air can be supplied to the discharge device, wherein this connection line is preferably also configured for electrically grounding at least one element of the discharge device and, in particular, an electrode.

15. The apparatus according to claim 1,
wherein
at least one parameter characteristic of the voltage used by the ionization unit for ionization is variable, wherein this parameter is preferably selected from a group of parameters including a frequency of the voltage, a maximum positive voltage, a maximum negative voltage, a temporal waveform of the voltage, a symmetry of the voltage with respect to a predetermined potential, and the like.

16. The apparatus according to claim 1,
wherein
the apparatus comprises a sensor device configured for detecting air pressure or an air flow.

17. The apparatus according to claim 1,
wherein
the apparatus comprises an lighting device configured for illuminating the surface to be treated, and in particular a automotive surface, wherein the lighting device preferably comprises a lens assembly , wherein, preferably, this lighting device comprises at least one optically diffracting element, which causes a surface illuminated by the lighting device to have a cross-section deviating from a circular cross-section and, in particular, an elliptical cross-section.

18. The apparatus according to claim 17,
wherein
the apparatus comprises a distance-measuring device that is configured to determine a distance between the apparatus and the surface to be treated.

19. The apparatus according to claim 5,
wherein
the lighting device is configured to be supplied with electrical energy by the energy conversion device.

20. The apparatus according to claim 1,
wherein
the apparatus comprises at least one ionization element formed with a tip and, preferably, at least two ionization elements formed with a tip, which ionize air flowing past these ionization elements.
